(19) United States
(12) Patent Application Publication
(10) Pub. No.: US 2025/0006479 A1
(43) Pub. Date: Jan. 2, 2025

(54) QUANTIFYING A SUBSTANCE PRESENT IN A SAMPLE

(71) Applicant: Thermo Fisher Scientific (Bremen) GmbH, Bremen (DE)

(72) Inventor: Pedro José Navarro Álvarez, Bremen (DE)

(73) Assignee: Thermo Fisher Scientific (Bremen) GmbH, Bremen (DE)

(21) Appl. No.: 18/758,170

(22) Filed: Jun. 28, 2024

(30) Foreign Application Priority Data
Jun. 30, 2023 (GB) 2310046.4

(51) Int. Cl. H01J 49/00; G01N 27/623; G01N 30/72; H01J 49/40

(52) U.S. Cl. CPC H01J 49/0036; G01N 27/623; G01N 30/72; H01J 49/40

(57) ABSTRACT

A sample is separated by a first separator into constituent analytes over a time parameter. The constituent analytes are analysed by a mass spectrometer, which provides intensity measurements against mass-to-charge ratio for each constituent analyte. A relationship of the measured intensity at a mass-to-charge ratio over the time parameter for each constituent analyte defines a respective peak. A peak quality factor may be determined for each of the peaks and/or a common peak position in respect of the time parameter may be determined for at least some of the peaks. A specific range for a time parameter to be used for quantifying the substance may be based on the peak quality factor (or factors). Alternatively, a subset of the peaks may be selected for quantification of the substance, based on the determined peak quality factors and/or the determined common peak position.

### CROSS REFERENCE TO RELATED APPLICATION

This application claims priority from application GB 2310046.4, filed Jun. 30, 2023. The entire disclosure of application GB 2310046.4 is incorporated herein by reference.

### TECHNICAL FIELD OF THE DISCLOSURE

The disclosure concerns quantifying a substance present in a sample, the sample being separated by a first separator (for example, a chromatographic or ion mobility separator) into constituent analytes over a time parameter, the constituent analytes then being further analysed by a mass spectrometer. This may be embodied in a method, a computer program, controller for a mass spectrometry system and/or a mass spectrometry system.

### BACKGROUND TO THE DISCLOSURE

Software analysis of mass spectrometry data is increasingly beneficial in identification and quantification of substances present in a sample. Analysis of MS1 data is generally considered more straightforward than analysis of MSⁿ data and analysis of Data Independent Acquisition (DIA) data can be even more complex. The mass analysis may follow an initial separation stage, for instance using a chromatographic separator, including gas chromatography-mass spectrometry (GC-MS) or liquid chromatography-mass spectrometry (LC-MS), or an ion mobility separator, commonly known as Ion-mobility spectrometry-mass spectrometry (IMS-MS). This only increases the complexity of workflow for analysis.

Conventionally, there are two main approaches to DIA data analysis. A first approach uses database-based search engines that are commonly used for analysis of Data Dependent Acquisition (DDA) data. A second option is targeted analysis, also known as SWATH-MS (Sequential Windowed Acquisition of All Theoretical Fragment Ion Mass Spectra). These data analysis algorithms are mainly focused on sample identification, but quantification is also considered.

WO-2009/146345 discusses matching a precursor ion with one or more related product ions. Data sets including the information in respect of the precursor ion are obtained from multiple injections and then normalized in accordance with a single retention time. By determining which product ions are within a predetermined retention time window with respect to the single retention time, the presence of such product ions allows their relationship with the precursor ion to be established.

WO-2012/035412 concerns the use of multiple product ions to characterize an unknown sample compound. The chromatographic peak retention time can be correlated across the product ions to improve identification.

Typical existing approaches towards quantification use a single raw data sample (or single event) basis. For example, Barkovits, Katalin, et al. “Reproducibility, specificity and accuracy of relative quantification using spectral library-based data-independent acquisition” (Molecular & Cellular Proteomics 19.1 (2020): 181-197) discusses selection of suitable spectral libraries for peptide identification and quantification. Searle, Brian C., et al. “Generating high quality libraries for DIA MS with empirically corrected peptide predictions” (Nature communications 11.1 (2020): 1-10) describes library generation using empirical data, such as fragmentation and retention time prediction. Demichev, Vadim, et al. “DIA-NN: neural networks and interference correction enable deep proteome coverage in high throughput” (Nature methods 17.1 (2020): 41-44) describes removing interfering product ions by choosing the best correlated chromatographic product ion peaks with their respective product ion partners.

It is therefore desirable to improve analysis of mass spectrometry data in order to provide more accurate quantification of a substance within a sample. This is especially the case where the sample is first separated into constituent analytes, each of which is then mass analysed.

### SUMMARY OF THE DISCLOSURE

Against this background, there are provided methods of quantifying a substance present in a sample according to claim 1 and according to claim 9, a computer program in line with claim 23, a controller for a mass spectrometry system as defined by claim 24 and a mass spectrometry system in accordance with claim 25. Preferred and/or optional features are disclosed in the dependent claims.

The present disclosure is concerned with quantifying a substance present in a sample. The sample is separated by a first separator into constituent analytes over a time parameter. For example, the first separator may be a chromatographic separator (GC or LC, for instance). In this case, the sample is typically a liquid or gas phase sample. Then, the time parameter relates to a retention time and the constituent analytes are separated components from the chromatographic separator at different retention times. In another example, the first separator is an ion mobility separator. Then, the sample is a mixture of sample ions. In this case, the time parameter relates to a drift time or a retention time and the constituent analytes are constituent ions of the mixture of sample ions.

Aspects according to the disclosure may be embodied in software (as a computer program, which may be stored on a computer readable medium, optionally non-transitory), in a controller for a mass spectrometry system and/or a mass spectrometry system. Additionally or alternatively, these aspects may be implemented as a process.

In any case, the constituent analytes are further analysed by a mass spectrometer (where the first separator is a chromatographic separator, each constituent analyte is initially ionised). For instance, analysis of the constituent analytes in the mass spectrometer may comprise: processing the ions; and mass analysing the processed ions. In some cases, the processing does not involve fragmentation (although it may involve mass filtering or selection and/or cooling) and the mass analysis may be of unfragmented ions. Alternatively, the ions may be precursor ions and the processing of the ions comprises fragmenting the precursor ions to produce fragment ions, such that the fragment ions are mass analysed. Then, the mass analysis of the fragment ions may comprise Data Independent Acquisition (DIA) MSⁿ analysis.

Measurements of intensity against mass-to-charge ratio are obtained from the mass spectrometer for each of one or more constituent analytes. Multiple such measurements are made across the time parameter. For each constituent analyte, a relationship of the measured intensity over the time parameter for a selected mass-to-charge ratio (or mass-to-charge ratio range) defines a peak. Preferably, each peak comprises at least a minimum number of intensity measurements, each for a different value of the time parameter (for example, at least 5 measurements).

In some cases, ions of the substance being quantified have one of the selected mass-to-charge ratios, which may allow application of the disclosed techniques to analysis of MS1 full scans, typically analysing multiple ions within a single scan (rather than quantification of a single peak). More commonly, each of the selected mass-to-charge ratios relates to a respective fragment of ions of the substance being quantified. Quantification may involve identifying the substance and/or identifying a chemical composition corresponding with one or more peaks (such that the selected mass-to-charge ratio or range of the peak or peaks corresponds with the identified chemical composition).

In a first aspect, there may be a single peak (that is, only data for a single mass-to-charge ratio or range is provided over the time parameter), but multiple peaks are more typically obtained. A peak quality for each peak is established and this is used to determine a specific range for the time parameter. The determined specific range for the time parameter can then be used to quantify the substance. It is particularly noted that the peak quality factor is dependent on a range for the time parameter used for establishing the peak quality factor.

A flatness detection algorithm may additionally be used (for example, as an algorithm or one of a plurality of algorithms for determining a peak quality factor) to determine the specific range for the time parameter.

The specific range for the time parameter can be determined iteratively. For example, a first range of the time parameter may be used to establish a first peak quality factor for each peak. Then, a second range of the time parameter is identified, which may be narrower or broader than the first range of the time parameter. For example, the second range may be based on a predetermined increase or decrease from the first range of the time parameter (at either or both ends of the range). Optionally, the second range may be based on the first peak quality factor (or factors), for instance by their comparison with a criterion (or criteria, for example one or more thresholds). The identified second range of the time parameter may be used to establish a second peak quality factor for each peak. The specific range for the time parameter may thus be based on the first peak quality factor (or factors) and the second peak quality factor (or factors). This procedure may be continued until the peak quality factor (or factors) meet the criterion (or criteria). For example, the range of the time parameter may be increasingly broad until the criterion (or criteria) are met.

The initially evaluated measurements of intensity against mass-to-charge ratio may be for the first range of the time parameter. If the second range of the time parameter is broader than the first range, additional measurements of intensity against mass-to-charge ratio may be evaluated for the constituent analyte (or analytes). In this case, the initial intensity measurements and additional intensity measurements may together cover the second range of the time parameter. The second peak quality factor (or factors) may thus be based on the initial intensity measurements and the additional intensity measurements.

The substance is advantageously quantified based on the intensity measurements for each of the one or more peaks falling within the determined specific range. The determined specific range for the time parameter may be used not only for quantification of the measured sample, but also of the same substance from a different sample. Alternatively, parallel evaluation of a specific range for the time parameter for each of multiple samples can be performed. In this case, a specific range for the time parameter may be established for the evaluated samples.

In a second aspect, multiple peaks of intensity measurement over the time parameter are obtained (each peak relating to a respective mass-to-charge ratio or range). A respective peak quality factor may be established for each peak (the peak quality factor depending on a range for the time parameter) and/or a common peak position may be determined in respect of the time parameter for at least some of the peaks. A subset of the peaks is then selected for quantification of the substance, based on the determined peak quality factors and/or the determined common peak position.

In one approach, the subset of the peaks may be selected by evaluating a peak position for each of the peaks against the determined common peak position. For example, only peaks having a peak position within a predetermined amount away from the determined common peak position may be selected for the subset.

In another approach (which may be used together with the approach discussed above), the subset of the peaks may be selected by evaluating the respective peak quality factor for each of the peaks against a criterion (for instance, being greater than, at least, equal to, no more than or less than a threshold). The subset of the peaks may then be selected based on whether the criterion is met for each peak (that is, selected peaks may be those that meet the criterion). Optionally, there may be multiple criteria.

Each of the selected subset of the peaks may then be quantified. The substance may then be quantified based on the quantification of the selected subset of the peaks.

Processes according to the disclosure need not use correlation of peak intensities among product ions nor correlation with a precursor mass.

It will be noted that the multiple aspects of the disclosure may be combined. According to any aspect, the mass spectrometer may be controlled to perform mass analysis of each of the constituent analytes and provide the intensity measurements against mass-to-charge ratio for each of the plurality of the components.

### BRIEF DESCRIPTION OF THE DRAWINGS

The disclosure may be put into practice in a number of ways, and preferred embodiments will now be described by way of example only and with reference to the accompanying drawings, in which:

FIG. 1 schematically depicts a mass spectrometry system for use in accordance with the disclosure;

FIG. 2 plots measured intensity against time peaks for three example mass-to-charge ratio ranges;

FIG. 3 shows a block diagram of a first procedure in accordance with the disclosure;

FIG. 4 shows a block diagram of a second procedure in accordance with the disclosure; and

FIGS. 5A-5D each illustrate a plot of measured intensity against time peaks, showing the effect of procedures in accordance with the disclosure.

### DETAILED DESCRIPTION OF PREFERRED EMBODIMENTS

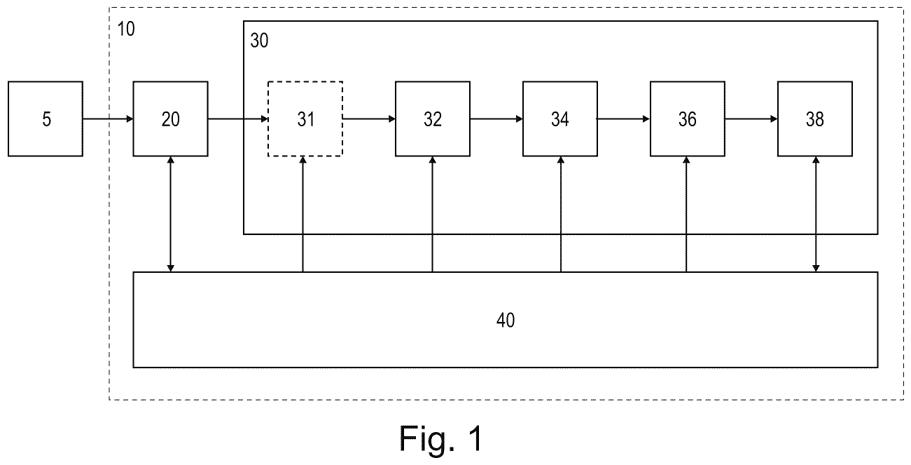

Referring first to FIG. 1, there is schematically depicted a mass spectrometry system for use in accordance with the disclosure. The mass spectrometry system 10 receives a sample 5 and comprises: a first separator 20; a mass spectrometer 30; and a controller 40. The mass spectrometer 30 comprises: at least one ion optical device 32 (for example, an ion guide, ion trap and/or ion selector or filter); a collision cell 34; a mass analyser 36; and a detector 38. Optionally, the mass analyser 36 and the detector 38 may be combined into a single device.

The first separator 20 separates the sample 5 into constituent analytes. This separation occurs over time, with different constituent analytes being output from the first separator 20 at different times. Thus, there is a time parameter associated with the first separator 20, each constituent analyte having a different corresponding value (or range) for the time parameter.

Preferably, the first separator 20 is a chromatographic separator, for instance a Gas Chromatography (GC) apparatus or Liquid Chromatography (LC) apparatus, such that the time parameter may be a Retention Time (RT). Then, the mass spectrometry system 10 may be a GC-MS or LC-MS system. In such cases (and optionally with other types of separator), the mass spectrometer 30 further comprises an ion source 31, advantageously configured to receive separated analytes (effluent or eluate) from the first separator 20 and generate ions from the received separated analytes.

Alternatively, the first separator 20 may be an Ion Mobility Spectrometer (IMS), such that the time parameter may be a drift time (for instance, in the case of drift-tube-type or travelling wave-type ion mobility separation) or a retention time (for example, in the case of trapped ion mobility separation, TIMS). Then, the mass spectrometry system 10 may be an IMS-MS system. The first separator 20 may then comprise an ion source (not shown) and the output of the first separator 20 in this case comprises ions. Hence, the mass spectrometer 30 need not comprise an ion source 31 and the ion source 31 is hence shown as optional in FIG. 1.

The controller 40 is configured to control operation of the first separator 20 and the mass spectrometer 30. In addition, the controller 40 receives mass spectral data from the detector 38. Optionally, the controller 40 may also receive data from the first separator 20. Thus, the controller may cause the mass spectrometry system 10 to run experiments and then receive the results of the experiment for analysis. The analysis may be performed by the controller or by an external processor or processing system (not shown). The controller 40 is typically a single device, but it may comprise multiple parts, which typically operate together.

As discussed above, approaches according to the disclosure are especially advantageous for analysis of mass spectral data from DIA MSⁿ (typically MS²) experiments. In this case (and controlled by the controller 40), the mass spectrometry system 10 is operated to separate the sample 5 into one or more constituent analytes, each having an associated range for the time parameter. Thus, a single constituent analyte may be output from the first separator 20 over a range of the time parameter, thus resulting in multiple outputs of constituent analytes from the first separator 20. Each output of a constituent analyte, having an associated time parameter is then provided to the mass spectrometer 30 where it is fragmented in collision cell 34, analysed by the mass analyser 36, with mass spectral data being detected by the detector 38. The mass spectral data is then communicated to the controller 40. Each mass analysis typically indicates multiple fragments, each fragment being at a different mass-to-charge ratio (m/z) and a having a corresponding detected intensity. This is referred to as an MSⁿ mass spectrum. As a result, the controller 40 receives multiple MSⁿ mass spectra, each MSⁿ mass spectrum relating to one or more output constituent analytes from the first separator 20 (and thus having an associated time parameter). Typically, multiple MSⁿ mass spectra are obtained for each single output constituent analyte. In other words, multiple MSⁿ mass spectra are obtained during the output of each constituent analyte.

Optionally, multiple MS1 mass spectra may also be obtained for each single output constituent analyte, that is multiple MS1 mass spectra may be obtained during the output of each constituent analyte.

Quantification is typically performed by first using a DIA identification tool on the mass spectra to make an initial identification of the constituent analyte compound (or compounds). Each compound identification provides a point of the time parameter (for example, a retention time point), where the compound has been detected and a list of detected product (that is, fragment) ions. This list may be exhaustive or just a selection of the most intense or accurate detected product (fragment) ions.

Then, recovering peaks from the MSⁿ analysis is possible by targeting specific m/z and time parameter values of the set of product (fragment) ions. For example, intensities may be obtained for the set of product (fragment) ions at their specific m/z values (optionally subject to a user-defined or automatically estimated tolerance) for the point of the time parameter determined by the DIA identification tool. Then, intensities for the same m/z values (again, optionally subject to user-defined or automatically estimated tolerance) may be retrieved for spectra at adjacent values of the time parameter. This retrieval process may then be repeated until a minimum number of spectra, min_s (typically, min_s=5) is reached. In so doing, it is possible to correlate or match up intensity measurements for the same fragment (based on m/z value or range) across the time parameter.

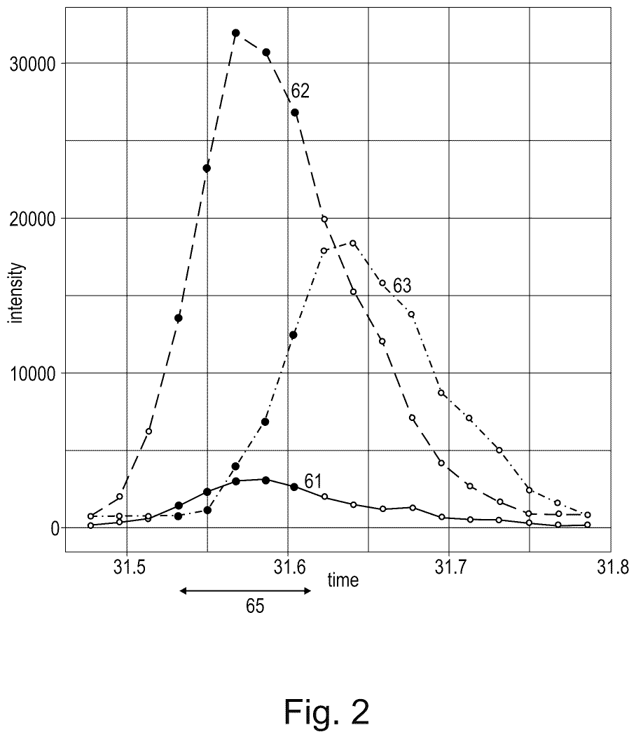

This data may be represented graphically. With reference to FIG. 2, there is plotted peaks of measured intensity against time for three example fragments (each fragment corresponding with a respective m/z range) from a DIA analysis. A first peak 61, corresponding with a first fragment, is relatively low (intensity approximately 3500), with a peak position at approximately 31.58 on the time axis. A second peak 62, corresponding with a second fragment, is relatively high (intensity approximately 32000), with a peak position at approximately 31.57 on the time axis. A third peak 63, corresponding with a third fragment, has an intermediate intensity (approximately 18000), with a peak position at approximately 31.64 on the time axis. All three peaks are present in approximately the same range of retention times.

It is desirable to use the information from these three peaks to quantify the substance from which the three fragments appear to have been derived. Quantification may be possible by summing the peak intensities for the identified product ions over a specific range of the time parameter. The same range is beneficially used for all peaks. Identification points to a relatively arbitrary point on the time parameter range (for instance, a specific time of the compound elution), which might not be the peak retention or even indicative of a suitable range.

Referring back to FIG. 2, a selected range 65 is shown from the time parameter of approximately 31.53 to approximately 31.62. The data points from the first peak 61, second peak 62 and third peak 63 falling within the selected range 65 are highlighted. Improved selection of the range 65 is thus desirable. The second peak 62 starts slightly earlier than the first peak 61. The third peak 63 starts at a noticeably later time than the first peak 61 and second peak 62. It can also be seen that the peak position of the third peak 63 is somewhat different from the peak position of the first peak 61 and second peak 62 (in other words, the third peak 63 is offset from the other two). It is therefore unclear whether the fragment corresponding with the third peak 63 is derived from the same substance (parent or precursor ion) as the respective fragments corresponding with the first peak 61 and the second peak 62.

It has been identified that a mathematical approach may be used to address these issues. In particular, a Peak Quality Factor (PQF) algorithm may be used to objectively and quantitatively evaluate the quality of each peak over the selected range for the time parameter. More typically, multiple PQF algorithms may be used, for instance as discussed in “MetaClean: a machine learning-based classifier for reduced false positive peak detection in untargeted LC-MS metabolomics data”, Chetnik et al., Metabolomics 16 (2020): 1-13. Selecting the range for the time parameter that maximises the result of the PQF algorithms may therefore allow improved quantification of the peaks and the substance.

Only data from a limited range of the time parameter may initially be provided to the quantification algorithm. The algorithm may therefore desirably retrieve ion product intensities from mass spectra of adjacent time parameter points. The PQF algorithms may also inform which direction to retrieve additional data (up and/or down in respect of the time parameter) and when to stop retrieving. This process may be run simultaneously over all identifications of the same compound in all samples of the experiment, which may ensure that the same set of product ions is used across all samples. This allows a product ion to be discarded from all samples when the output of the PQF algorithms determines that the peak for the product ion is not of sufficient (user-defined) quality on any of the samples. This also allows to define a fixed range (or at least width) of the time parameter for all samples, if required by the user (in other words, the same width or range used for multiple samples).

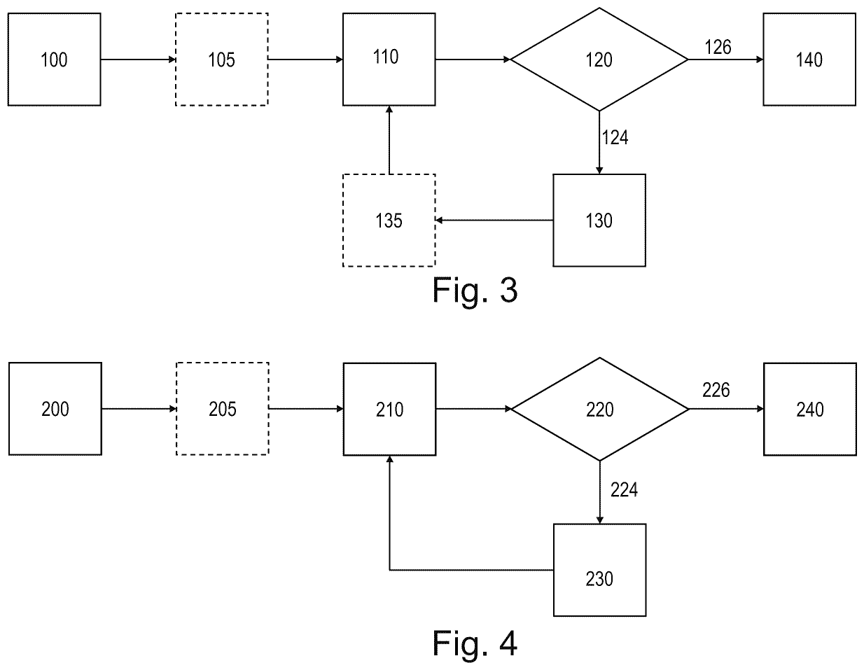

Referring now to FIG. 3, there is shown a block diagram of a first procedure in accordance with the disclosure. This procedure is specifically for selecting a range for the time parameter for the purposes of substance quantification. In an initial step 100, the mass spectrometer provides (or a controller retrieves from the mass spectrometer) data comprising intensity measurements against mass-to-charge ratio for each of one or more constituent analytes. As noted above, a relationship of the measured intensity at each selected mass-to-charge ratio over the time parameter for each constituent analyte defines a respective peak (as shown in FIG. 2). Typically, the data is obtained from a DIA experiment, in which multiple MS² spectra have been obtained as a function of a time parameter, for instance a Retention Time (RT) for GC-MS or LC-MS. As a result, there are typically multiple peaks (each for a different m/z value or range), each peak corresponding with a specific fragment of one of the constituent analytes.

Advantageously, an optional identification step 105 is run first. This uses a (known) identification algorithm that processes the received data in order to determine the identity of the compounds in the data. Each identification may preferably include a list of the m/z ratios of plural (n) product or fragment ions of the identified parent ion. Known algorithms typically allow each identification to be made within any one (but normally only one) of the MS² spectra. The MS² spectrum used is not necessarily the one corresponding to the apex of the intensity peak over time. Typically, an identification is made in the MS² spectrum at an arbitrarily selected value for the time parameter, which may be termed a base time, to. The identified parent ion (and its corresponding originating compound) is therefore assumed to include all fragments identified at that value of the time parameter.

In PQF step 110, one or more PQF algorithms are performed on the retrieved data aggregated across a selected range of the time parameter, to determine a PQF result. The selected range desirably includes a minimum number of data points, generally at least 5 data points. To achieve this, the selected range is typically set to include: the base time, t₀; two data points before the base time, t₀, that is at t₋₁ and t₋₂; and two data points after the base time, t₀, that is at t₁ and t₂. Any of the peaks for which the intensity is below a predetermined threshold for these first minimum number of data points (for example, 5) are preferably discarded, because in general, a minimum number of data points are needed to evaluate the peak properties for a fragment.

The PQF result is then compared with at least one PQF criterion in verification step 120. The one or more PQF algorithms may thus determine if each peak shape is good enough or if a product ion is not producing a good peak for quantification. A flatness detector algorithm may form part of the PQF algorithms, to determine when a peak boundary in either or both directions is reached. Thus, the at least one PQF criterion may comprise determining if a threshold minimum number of peaks, min_peaks, of enough quality (according to the PQF result) are available for quantification. This threshold may be user-defined or automatically defined. Another criterion may concern optimization (for instance, maximization) of the PQF result.

If the at least one PQF criterion is not satisfied, then range change path 124 is taken to range change step 130. In range change step 130, it is determined how to change the selected range of the time parameter. For example, the selected range of the time parameter may be extended at one or both ends of the range (for instance, to include t₋₃ and/or t₃), reduced at one or both ends of the range or extended at one end and reduced at the other end. The use of multiple PQF algorithms may allow such decisions to be made. The selected range of the time parameter is then updated accordingly. As noted above, a minimum number of data points are needed to evaluate the peak properties for a fragment. Thus, if the change in the selected range results in a fragment having insufficient data points above the threshold level to meet the minimum, the fragment is preferably disregarded.

Based on the updated selected range of the time parameter, additional data may be retrieved. This is done in retrieval step 135. It will be appreciated that this may not always be required, so this step is optional (indicated by a dashed outline).

The process then continues by repeating PQF step 110, but now with the updated selected range of the time parameter. It will be understood that this loop may repeat multiple times if the at least one PQF criterion considered in verification step 120 is not satisfied. It is expected that the selected range may be adjusted both forward and backwards in time with reference to the base time, to.

If the at least one PQF criterion considered in verification step 120 is satisfied (for instance, the PQF result is optimised and/or any other criterion or criteria are met), quantification path 126 is taken. In quantification step 140, the substance is quantified based on the selected range of the time parameter (if appropriate, as updated in immediately preceding range change step 130). This may be achieved by summing the intensities of the remaining fragments between the boundaries defined by the updated selected time range.

The above approach focuses on determining the range of the time parameter for use in quantification. The fragments selected for inclusion in the quantification are also considered. However, this is not the main focus of this approach. In another approach, which is preferably combined with the approach discussed above, the main focus is which fragments to include in quantification.

In general terms, there may be considered a method of quantifying a substance present in a sample. The sample is separated by a first separator into constituent analytes over a time parameter. The constituent analytes are then further analysed by a mass spectrometer. In practice, one constituent analyte may be provided (output) from the first separator over a range of the time parameter (over a time range). Thus, the mass spectrometer may provide multiple mass spectra in respect of each constituent analyte.

In one example, the sample comprises (or is) a liquid or gas phase sample. Then, the first separator may be a chromatographic separator and the time parameter relates to a retention time. In this case, the constituent analytes are separated components from the chromatographic separator at different retention times and the constituent analytes are further analysed by the mass spectrometer by ionising each analyte to provide ions. The constituent analytes, which are ionised to provide the ions, are then analysed in the mass spectrometer by: processing the ions, in particular by fragmentation; and mass analysing the processed (fragmented) ions.

Alternatively, the sample comprises (or is) a mixture of sample ions and the first separator is an ion mobility separator. Then, the time parameter relates to a drift time or a retention time and the constituent analytes are constituent ions of the mixture of sample ions. These may also be processed (for instance, by fragmentation) and the processed (fragmented) ions then mass analysed.

In any case, each of the peaks may typically correspond with a respective fragment ion from DIA MSⁿ (for example, MS²) analysis of the constituent analytes.

In a method according to a first aspect, intensity measurements against mass-to-charge ratio for each of one or more constituent analytes are received from the mass spectrometer. A relationship of the measured intensity at each of one or more selected mass-to-charge ratios over the time parameter for each constituent analyte defines a respective peak. In embodiments, the selected mass-to-charge ratios (which may be a range of mass-to-charge ratios) may be determined based on the fragments. Then, a specific range for the time parameter to be used for quantifying the substance is determined, based on a respective peak quality factor for each of the one or more peaks, the peak quality factor depending on a range for the time parameter. Typically, multiple peak quality factors may be used, each of which may provide a statistical measure of the peak, for example in terms of peak definition, symmetry, or other shape characteristics.

Preferably, the substance and/or a chemical composition corresponding with at least one peak (and typically each peak) are identified (after receiving the measurement data). In the latter case, the selected mass-to-charge ratio or mass-to-charge ratios of the at least one peak may correspond with the identified chemical composition. Identification of the peaks and/or substance may assist in determining a starting point and/or an initial range for the time parameter for each peak.

Beneficially, each peak comprises at least a predetermined number of intensity measurements, typically at least 5 intensity measurements (but optionally, 7, 9 or 11). Peaks having fewer than the predetermined number of intensity measurements may be disregarded.

Optionally, the specific range for the time parameter may be further determined using a flatness detection algorithm. This may allow determination as to whether the full extent of each peak is captured by the selected range for the time parameter.

In some implementations, determining the specific range for the time parameter comprises: establishing a respective first peak quality factor for each of the one or more peaks in relation to a first range of the time parameter; and establishing a respective second peak quality factor for each of the one or more peaks in relation to a second range of the time parameter, the second range of the time parameter being narrower or broader than the first range of the time parameter. Then, the specific range for the time parameter to be used for quantifying the substance may be determined based on the one or more first peak quality factors and the one or more second peak quality factors. For example, the change between the one or more second peak quality factors and the one or more first peak quality factors may indicate whether the specific range for the time parameter should be the first range of the time parameter, the second range of the time parameter, lower than the first range of the time parameter, higher than the second range of the time parameter or between the first range of the time parameter and the second range of the time parameter.

Advantageously, the second range of the time parameter may be selected based on the one or more first peak quality factors (for example, increasing the second range of the time parameter compared with the first range of the time parameter if the one or more first peak quality factors are too low) and/or based on a predetermined differential factor (for instance, a step increase or decrease in the second range of the time parameter compared with the first range of the time parameter).

Not all the data required to determine the one or more second peak quality factors may be initially evaluated. For example, the received intensity measurements against mass-to-charge ratio that are for each of the one or more constituent analytes may be initial intensity measurements for the first range of the time parameter. Then, where the second range of the time parameter is broader than the first range of the time parameter, additional intensity measurements against mass-to-charge ratio for the one or more constituent analytes may be evaluated. In this case, the initial intensity measurements and additional intensity measurements may together cover the second range of the time parameter. Then, the respective second peak quality factor for each of the one or more peaks may be established based on the initial intensity measurements and the additional intensity measurements.

In preferred embodiments, the specific range for the time parameter is determined iteratively by repeating the step of establishing a respective second peak quality factor for each of the one or more peaks in relation to a second range of the time parameter. Preferably, each second range of the time parameter is increasingly broad (so that the range of the time parameter is iteratively increased until the peak quality factor is found to meet the set criterion or criteria).

The specific range for the time parameter may be determined by evaluating the respective peak quality factor for each of the one or more peaks against a criterion (or criteria). The specific range for the time parameter may be determined based on the criterion being met.

Preferably, the substance is quantified based on (summing) the intensity measurements for each of the one or more peaks falling within the determined specific range.

Optionally, the determined specific range for the time parameter may be used for quantification of the same substance from a different sample.

Referring now to FIG. 4, there is shown a block diagram of a second procedure, along these lines. This procedure is specifically for determining the fragments to be included for the purposes of substance quantification. In an initial step 200, the mass spectrometer provides (or a controller retrieves from the mass spectrometer) data comprising intensity measurements against mass-to-charge ratio for each of one or more constituent analytes. In this case, there are necessarily multiple peaks (each for a different m/z value or range). This will typically occur when fragments (from DIA MS², for instance) of one of the constituent analytes are being considered, but there may be other situations where multiple peaks are being considered for quantification of a substance.

Advantageously, an optional identification step 205 is run first. This is discussed above with reference to identification step 105 in FIG. 3, so this is not repeated here for brevity. Either based on the initial step 200 or based on the optional identification step 205, an initial set of peaks are identified for quantification.

In evaluation step 210, an evaluation of the peaks identified for quantification is carried out. There are two options for this evaluation. A first option is for one or more PQF algorithms to be performed on the retrieved data aggregated across a range of the time parameter. A second option is for a common peak position to be determined. This may be established by a statistical measure, for example determining an average, which may include a mean, median or mode of the peak positions. Both options may be used together.

The evaluation result is then compared with at least one criterion in verification step 220. For example, the results of the PQF algorithm (or algorithms) for each peak may be compared against a threshold. Additionally or alternatively, the peak positions of each of the peaks may be compared against the determined common peak position. A threshold may be predetermined for the spacing between a peak position and the determined common peak position, such that a criterion may comprise the spacing exceeding the threshold. For example, one of the peaks may be an interfering peak derived from another parent ion. The separation in the time parameter domain may be sufficient to recognise that the peak should be excluded from quantification.

If the at least one criterion is not satisfied, then peak reselection path 224 is taken to peak removal step 230. In peak removal step 230, at least one peak from those previously identified for quantification is selected to be disregarded. This is typically determined based on the evaluation step 210. For example, if a peak does not meet a criterion (threshold) based on PQF and/or has a peak position that is at least (or more than) a threshold away from the determined common peak position, the peak is selected to be disregarded from the set of peaks identified for quantification.

The process then continues by repeating evaluation step 210, but now with the updated set of peaks identified for quantification. It will be understood that this loop may repeat multiple times if the at least one criterion considered in verification step 220 is not satisfied.

If the at least one criterion considered in verification step 220 is satisfied (for instance, all the peaks have a sufficient PQF and/or the peak position for all the peaks is no more (or less) than a threshold from the determined common peak position), quantification path 226 is taken. In quantification step 240, the substance is quantified based on the set of peaks identified for quantification as updated during the process. This may be achieved by summing the intensities of the fragments in the set of peaks identified for quantification, in particular between boundaries defined by a selected time range.

Advantageously, both two processes may be performed, simultaneously or in sequence. As a result, both a selected time range and a selected set of peaks may be determined for quantification purposes.

In either or both approaches, the result (selected time range and/or selected set of peaks) used for quantification of one sample are beneficially used for quantification of the same substance in another sample. For example, this may apply if multiple sample replicates are processed in multiple experimental runs. This may ensure consistent quantification between runs.

Returning to the general terms discussed above, another aspect may be considered (which may be combined with any other aspect disclosed herein). In this aspect, there may also be considered a method of quantifying a substance present in a sample. The same considerations as discussed above may apply to this aspect.

In the method, intensity measurements against mass-to-charge ratio for each of one or more constituent analytes are received from the mass spectrometer. A relationship of the measured intensity at each of a plurality of selected mass-to-charge ratios over the time parameter for each constituent analyte defines a respective peak. In other words, peaks in respect of a plurality of selected mass-to-charge ratios are received. For example, these may each represent fragments from the same substance (in DIA MSⁿ experiments, for instance).

A respective peak quality factor may be determined for each of the peaks. The peak quality factor depends on a range for the time parameter. Preferably, multiple peak quality factors may be determined for each of the peaks. Further details in terms of optional aspects and/or implementation of peak quality factor determination have been discussed above and apply equally here. Additionally or alternatively, a common peak position in respect of the time parameter is determined for at least some of the peaks. Then, a subset of the peaks is selected for quantification of the substance, based on the determined peak quality factors and/or the determined common peak position.

In this way, the peaks selected for quantification of the substance are determined based on their properties, specifically the quality of the peak and/or whether the peak are aligned by a common (maximal) position. The common peak position may be determined statistically, for instance by an averaging, weighted averaging or heuristic algorithm. Peaks of poor quality and/or offset peaks may be indicative of interferences and disregarding such peaks in the substance quantification may improve accuracy.

Optionally, selecting the subset of the peaks may comprise evaluating a peak position for each of the peaks against the determined common peak position (for example, determining an offset). Then, the subset of peaks is advantageously selected based on the evaluating (for instance, by disregarding any peaks with a peak position at least or more than a threshold away from the determined common peak position).

Advantageously, selecting the subset of the peaks comprises evaluating the respective peak quality factor (or factors) for each of the peaks against a criterion (for instance, whether one or more thresholds is met or exceeded, for example, each threshold may apply to a respective peak quality factor). Then, the subset of the peaks may be selected based on whether the criterion is met for each peak. There may be multiple criteria, each of which may apply to a respective peak quality factor.

Each of the selected subset of the peaks is preferably quantified (by summing the measured intensities across a selected range for the time parameter). Then, the substance may be quantified based on the quantification of the selected subset of the peaks (for example, summing them).

Preferably, the substance and/or a chemical composition corresponding with at least one peak (and typically each peak) are identified (after receiving the measurement data). This may further assist in determining a set of peaks from which the subset is to be selected.

As above, each peak beneficially comprises at least a predetermined number of intensity measurements, typically at least 5 intensity measurements (but optionally, 7, 9 or 11). Peaks having fewer than the predetermined number of intensity measurements may be disregarded.

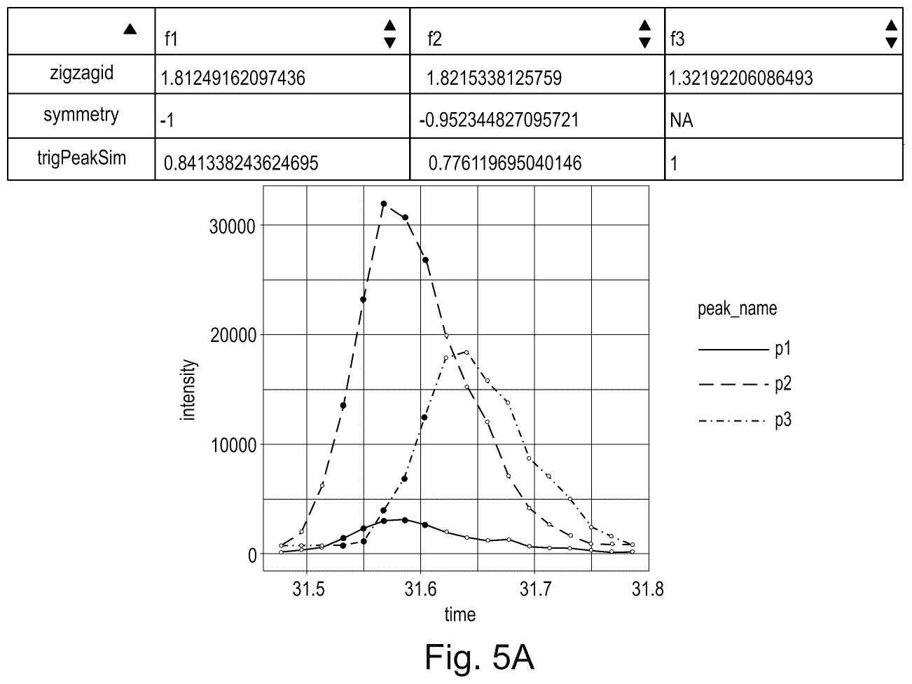

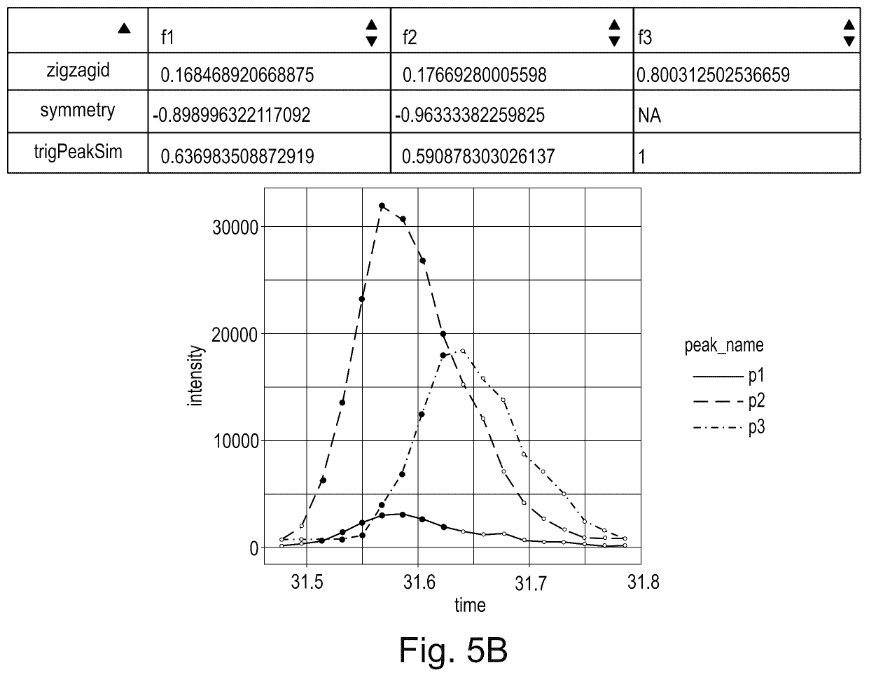

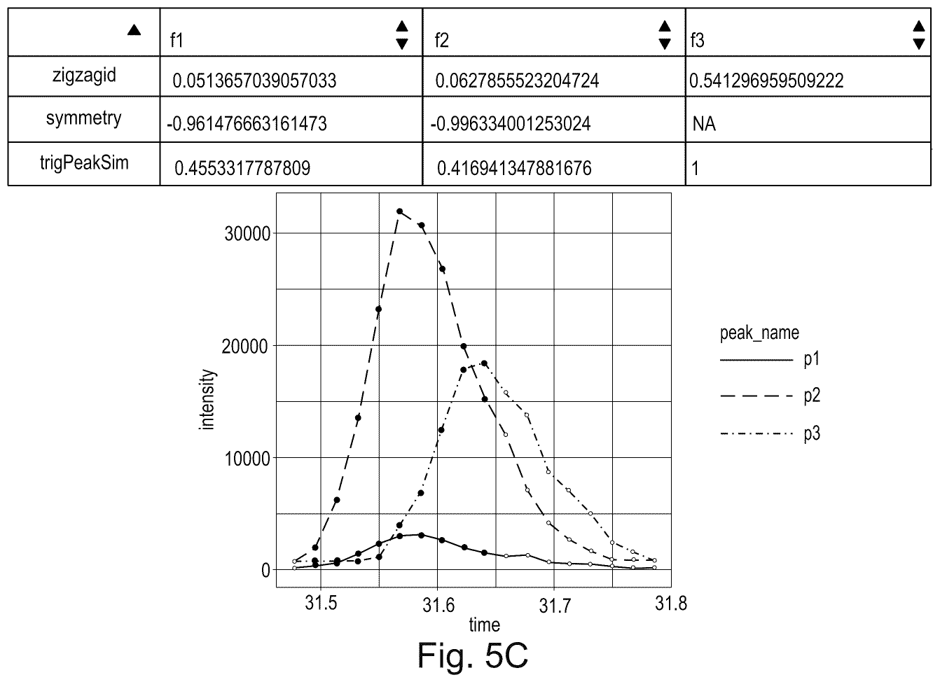

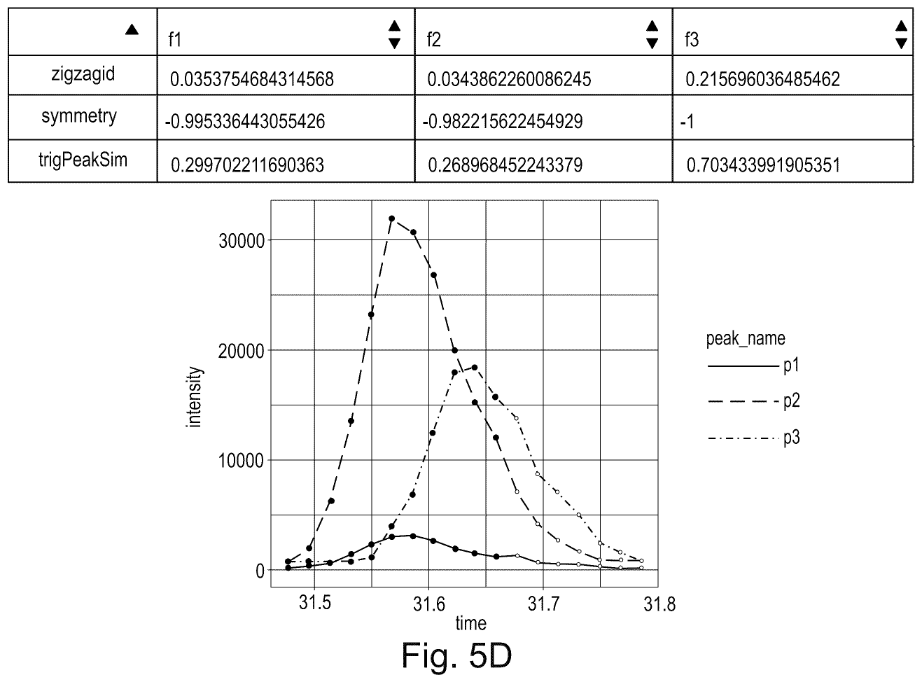

Referring now to FIGS. 5A-5D, these each illustrate a plot of measured intensity against time peaks, showing the effect of different PQF algorithms, when the time range for those algorithms is changed. These use the same data shown in FIG. 2, as discussed above, with three peaks. Three example PQF algorithms are used: ZigZagId (a smaller number is considered better); Symmetry (−1 is optimal and the closer to this value, the better); and trigPeakSim (1 is optimal and the closer to this value, the better).

A brief description of these example PQF algorithms is provided now for completeness (more complete details may be found in “MetaClean: a machine learning-based classifier for reduced false positive peak detection in untargeted LC-MS metabolomics data”, Chetnik et al., detailed above). ZigZagId is a shortened name for Zig-Zag index. A Zig-Zag Index (ZZ) captures shape quality by measuring the normalized variance between a point and its immediate neighbour on either side. A normalized average of all zig-zag index values (for every point of the peak except the two extremes, a zig-zag index value can be calculated) is the value used as quality factor for the peak. Where ZZ is the Zig-Zag index of the peak and Iₙ is the n^{th} intensity measurement (there being N measurements in the peak), this may be mathematically expressed as:

ZZ=ZZ1/ZZ2;

ZZ1=sum((2Iₙ−Iₙ₋₁−Iₙ₊₁), from n=2 to n=N−1;

ZZ2=N*EPI²; and

EPI=I_{A}−avg(I₁+I₂+I_{N-1}+I_{N}), where I_{A} is the value of the maximum intensity.

Symmetry (SY) measures correlation between left and right halves of a peak. This may be mathematically expressed as:

SY=cor([I1, . . . ,IN/2],[IN/2, . . . ,IN]), range: [−1,1].

TrigPeakSim (TPASR) is a shortened name for triangle peak area symmetry or similarity ratio. This peak quality factor estimates shape quality by comparing peak area to area of triangle formed by the apex and boundaries. This may be mathematically expressed as:

TPASR=abs(triangle_area−AUC)/max(triangle_area,AUC), where AUC is the area under the curve of the peak (or simply the peak area).

In FIG. 5A, five data points are selected as highlighted, in FIG. 5B, the time range is extended to seven data points, FIG. 5C uses nine data points and FIG. 5D selects eleven data points. For both the first peak (having the lowest peak intensity) and the second peak (having the highest peak intensity), it can be seen that the ZigZagId value and the trigPeakSim value decrease as the number of data points increase. The Symmetry value for the first and second peaks changes, but not significantly. For the third peak (having intermediate peak intensity and slightly offset in peak position compared with the other two peaks), the ZigZagId value decreases as the number of data points increase. However, the Symmetry value is indeterminate until the number of data points reaches 11. In contrast, the trigPeakSim value appears optimal with the number of data points as 5, 7 or 9, but lower when the number of data points reaches 11.

It will therefore be understood that a combination of PQF algorithms are beneficially used to evaluate the peaks and determine the number of data points (the selected range for the time parameter).

Returning to the general terms discussed above (in any aspect), it may be understood that the mass spectrometer may be controlled to perform mass analysis of each of the constituent analytes and provide the intensity measurements against mass-to-charge ratio for each of the plurality of the components.

Aspects according to the disclosure may be embodied as a computer program (optionally on a computer readable medium, which may be non-transitory), as a controller for a mass spectrometry system (in hardware and/or software) or as a mass spectrometry system comprising such a controller. It will be understood that such a mass spectrometry system may further comprise: a first separator, configured to separate a sample into constituent analytes over a time parameter; and a mass spectrometer, configured to receive and analyse the constituent analytes.

Processes, methods or implementations according to the disclosure may consume a minimum of RAM memory, since the minimum number of intensities are retrieved that may be necessary or desired in order to have a reliable quantification. Such processes may be run for multiple samples simultaneously. This may allow controlling consistency of product ion set utilized for quantification and similar peak boundaries for all samples.

The separation of DIA identification and DIA quantification may allow a second validation step, improving quantification and even discarding quantification of unreliable compounds (in case of all product ions from the common set of product ions in a sample failed to produce any reliable peak). Also, a (more) consistent quantification may be provided, since the same set of product ions can be used across all samples. Moreover, if any product ion failed to produce a reliable peak, the product ion can be removed from the common product ion set for all samples. Separating identification from quantification may particularly give the chance of evaluating the best and most common product ions set for each identified compound across all samples involved.

Although embodiments according to the disclosure have been described with reference to particular types of devices and applications (particularly mass spectrometry) and the embodiments have particular advantages in such case, as discussed herein, approaches according to the disclosure may be applied to other types of device and/or application. In particular, the devices according to the disclosure may be used for other applications. The specific structure, arrangement and operational details (for example, parameters) of the processes described, whilst potentially advantageous (especially in view of known configurations and capabilities), may be varied significantly to arrive at modes of operation with similar or identical performance. Other types of separation may be considered from those disclosed herein. Certain features may be omitted or substituted, for example as indicated herein. Each feature disclosed in this specification, unless stated otherwise, may be replaced by alternative features serving the same, equivalent or similar purpose. Thus, unless stated otherwise, each feature disclosed is one example only of a generic series of equivalent or similar features.

It has been suggested above that a retention time range (or equivalently, drift or other suitable time range) may be determined based on measurements of one sample and then used for another sample, especially if multiple sample replicates are processed in multiple experimental runs. Alternative approaches are possible. For instance, parallel evaluation of the retention time range may be made for all (or at least multiple) samples. This may allow a consensual (which may be a common or statistically determined, for instance average or weighted mean) retention time range for the evaluated samples to be determined. Where a parallel analysis of multiple samples is used, peaks having fewer than a predetermined number of intensity measurements need not be disregarded, but may be integrated into the quantification measurement. The determined time range may then be used for quantification of all samples (perhaps including other samples, not used for determining the range).

A variant of the methods described above is to apply the same algorithm to MS1 full scans, searching only the expected (precursor) mass for each identified compound. In this case, the term fragment used above may be replaced by identified compound and the process may be used in the same way.

In the general terms discussed above, it may optionally be considered that ions of the substance being quantified have one of the selected mass-to-charge ratios. Then, quantification of a peak may correspond with quantification of the substance.

In this detailed description of the various embodiments, for the purposes of explanation, numerous specific details are set forth to provide a thorough understanding of the embodiments disclosed. One skilled in the art will appreciate, however, that these various embodiments may be practiced with or without these specific details. Furthermore, one skilled in the art can readily appreciate that the specific sequences in which methods are presented and performed are illustrative and it is contemplated that the sequences can be varied and still remain within the scope of the various embodiments disclosed herein.

As used herein, including in the claims, unless the context indicates otherwise, singular forms of the terms herein are to be construed as including the plural form and vice versa. For instance, unless the context indicates otherwise, a singular reference herein including in the claims, such as “a” or “an” (such as an ion multipole device) means “one or more” (for instance, one or more ion multipole device). Throughout the description and claims of this disclosure, the words “comprise”, “including”, “having” and “contain” and variations of the words, for example “comprising” and “comprises” or similar, mean “including but not limited to”, and are not intended to (and do not) exclude other components. Also, the use of “or” is inclusive, such that the phrase “A or B” is true when “A” is true, “B is true”, or both “A” and “B” are true.

The use of any and all examples, or exemplary language (“for instance”, “such as”, “for example” and like language) provided herein, is intended merely to better illustrate the disclosure and does not indicate a limitation on the scope of the disclosure unless otherwise claimed. No language in the specification should be construed as indicating any non-claimed element as essential to the practice of the disclosure.

The terms “first” and “second” may be reversed without changing the scope of the disclosure. That is, an element termed a “first” element may instead be termed a “second” element and an element termed a “second” element may instead be considered a “first” element.

Any steps described in this specification may be performed in any order or simultaneously unless stated or the context requires otherwise. Moreover, where a step is described as being performed after a step, this does not preclude intervening steps being performed.

It is also to be understood that, for any given component or embodiment described herein, any of the possible candidates or alternatives listed for that component may generally be used individually or in combination with one another, unless implicitly or explicitly understood or stated otherwise. It will be understood that any list of such candidates or alternatives is merely illustrative, not limiting, unless implicitly or explicitly understood or stated otherwise.

All literature and similar materials cited in this disclosure, including but not limited to patents, patent applications, articles, books, treaties and internet web pages are expressly incorporated by reference in their entirety for any purpose. Unless otherwise described, all technical and scientific terms used herein have a meaning as is commonly understood by one of ordinary skill in the art to which the various embodiments described herein belongs.

1. A method of quantifying a substance present in a sample, the sample being separated by a first separator into constituent analytes over a time parameter, the constituent analytes then being further analysed by a mass spectrometer, the method comprising:
receiving, from the mass spectrometer, intensity measurements against mass-to-charge ratio for each of one or more constituent analytes, a relationship of the measured intensity at each of one or more selected mass-to-charge ratios over the time parameter for each constituent analyte defining a respective peak; and
determining a specific range for the time parameter to be used for quantifying the substance based on a respective peak quality factor for each of the one or more peaks, the peak quality factor depending on a range for the time parameter.

2. The method of claim 1, wherein the specific range for the time parameter is further determined using a flatness detection algorithm.

3. The method of claim 1, wherein determining the specific range for the time parameter comprises:
establishing a respective first peak quality factor for each of the one or more peaks in relation to a first range of the time parameter; and
establishing a respective second peak quality factor for each of the one or more peaks in relation to a second range of the time parameter, the second range of the time parameter being narrower or broader than the first range of the time parameter,
wherein the specific range for the time parameter to be used for quantifying the substance is determined based on the one or more first peak quality factors and the one or more second peak quality factors.

4. The method of claim 3, wherein the second range of the time parameter is selected based on the one or more first peak quality factors and/or a predetermined differential factor.

5. The method of claim 3, wherein the received intensity measurements against mass-to-charge ratio for each of the one or more constituent analytes are initial intensity measurements for the first range of the time parameter and the second range of the time parameter is broader than the first range of the time parameter, the method further comprising:
receiving additional intensity measurements against mass-to-charge ratio for the one or more constituent analytes, the initial intensity measurements and additional intensity measurements together covering the second range of the time parameter; and
wherein the respective second peak quality factor for each of the one or more peaks is established based on the initial intensity measurements and the additional intensity measurements.

6. The method of claim 3, wherein the specific range for the time parameter is determined iteratively by repeating the step of establishing a respective second peak quality factor for each of the one or more peaks in relation to a second range of the time parameter, each second range of the time parameter being increasingly broad.

7. The method of claim 1, further comprising:
using the determined specific range for the time parameter for quantification of the same substance from a different sample; and/or
quantifying the substance based on the intensity measurements for each of the one or more peaks falling within the determined specific range.

8. The method of claim 1, wherein the step of determining the specific range for the time parameter comprises:
evaluating the respective peak quality factor for each of the one or more peaks against a criterion, the specific range for the time parameter being determined based on the criterion being met.

9. A method of quantifying a substance present in a sample, the sample being separated by a first separator into constituent analytes over a time parameter, the constituent analytes then being further analysed by a mass spectrometer, the method comprising:
receiving, from the mass spectrometer, intensity measurements against mass-to-charge ratio for each of one or more constituent analytes, a relationship of the measured intensity at each of a plurality of selected mass-to-charge ratios over the time parameter for each constituent analyte defining a respective peak;
determining a respective peak quality factor for each of the peaks, the peak quality factor depending on a range for the time parameter and/or determining a common peak position in respect of the time parameter for at least some of the peaks; and
selecting a subset of the peaks for quantification of the substance, based on the determined peak quality factors and/or the determined common peak position.

10. The method of claim 9, wherein selecting the subset of the peaks comprises:
evaluating a peak position for each of the peaks against the determined common peak position, the subset of peaks being selected based on the evaluating.

11. The method of claim 9, wherein selecting the subset of the peaks comprises:
evaluating the respective peak quality factor for each of the peaks against a criterion, the subset of the peaks being selected based on whether the criterion is met for each peak.

12. The method of claim 9, further comprising:
quantifying each of the selected subset of the peaks; and
quantifying the substance based on the quantification of the selected subset of the peaks.

13. The method of claim 9, further comprising:
determining a specific range for the time parameter to be used for quantifying the substance based on a respective peak quality factor for each of the one or more peaks, the peak quality factor depending on a range for the time parameter.

14. The method of claim 11, wherein ions of the substance being quantified have one of the selected mass-to-charge ratios.

15. The method of claim 9, wherein the constituent analytes are analysed in the mass spectrometer as ions by: processing the ions; and mass analysing the processed ions.

16. The method of claim 15, wherein the sample is a liquid or gas phase sample, the first separator is a chromatographic separator, the time parameter relates to a retention time, the constituent analytes are separated components from the chromatographic separator at different retention times and the constituent analytes are further analysed by the mass spectrometer by ionising each analyte to provide the ions.

17. The method of claim 15, wherein the sample is a mixture of sample ions, the first separator is an ion mobility separator, the time parameter relates to a drift time or a retention time, the constituent analytes being constituent ions of the mixture of sample ions.

18. The method of claim 15, wherein the ions are precursor ions, and the processing of the ions comprises fragmenting the precursor ions to produce fragment ions, such that the fragment ions are mass analysed.

19. The method of claim 18, wherein each of the peaks corresponds with a respective fragment ion from Data Independent Acquisition (DIA) MSⁿ analysis of the constituent analytes.

20. The method of claim 9, further comprising:
identifying the substance; and/or
identifying a chemical composition corresponding with at least one peak, the selected mass-to-charge ratio or mass-to-charge ratios of the at least one peak corresponding with the identified chemical composition.

21. The method of claim 9, wherein each peak comprises at least a predetermined number of intensity measurements.

22. The method of claim 16, further comprising:
controlling the mass spectrometer to perform mass analysis of each of the constituent analytes and provide the intensity measurements against mass-to-charge ratio for each of the plurality of the separated components.

23. At least one non-transitory computer-readable media having stored thereon processor-executable instructions for performing the method of claim 1.

24. A controller for a mass spectrometry system, configured to control the mass spectrometry system to perform the method of claim 1.

25. A mass spectrometry system, comprising:
a first separator, configured to separate a sample into constituent analytes over a time parameter;
a mass spectrometer, configured to receive and analyse the constituent analytes; and
a controller configured to control the mass spectrometry system to perform the method of claim 1.
